(19) United States
(12) Patent Application Publication
(10) Pub. No.: US 2006/0194883 A1
(43) Pub. Date: Aug. 31, 2006

(54) Mmp expression inhibitor

(75) Inventors: Iwata Ozaki, Saga (JP); Hao Zhang, Saga (JP); Toshihiko Mizuta, Saga (JP); Kyosuke Yamamoto, Saga (JP)

Correspondence Address: STERNE, KESSLER, GOLDSTEIN & FOX PLLC, 1100 NEW YORK AVENUE, N.W., WASHINGTON, DC, 20005, US

(73) Assignee: EISAI CO., LTD

(21) Appl. No.: 10/553,434

(22) PCT Filed: Apr. 23, 2004
(86) PCT No.: PCT/JP04/06038

§ 371 Date: Oct. 18, 2005

(30) Foreign Application Priority Data
Apr. 23, 2003 (JP) 2003-118581

(51) Int. Cl. A61K 31/12

(52) U.S. Cl. 514/682

(57) ABSTRACT

An extracellular matrix protease (MMP) is an enzyme which is expressed in the course of tissue generation and differentiation via repeated cell division of a fertilized egg, and is also closely associated with invasion and metastasis of cancer. Degradation of extracellular matrix around cancer cells and in the vascular basal membrane is a necessary process for invasion and metastasis of cancer. Thus, invasion and metastasis of cancer can be prevented by inhibiting the expression of MMP, but few highly safe drugs that inhibit the expression of MMP have been developed. The present invention provides a drug that inhibits expression of MMP by cancer cells.

### TECHNICAL FIELD

The present invention relates to a medicament which inhibits the expression of an extracellular matrix protease (MMP: matrix metalloproteinase) on cancer cells.

### BACKGROUND ART

Extracellular matrix (sometimes abbreviated as ECM) is a general term for the insoluble components which immobilize and adhere the various cells which make up multicellular organisms. Extracellular matrix is known to affect proliferation and differentiation of cells via cell adhesion, and includes principally collagen, fibronectin, laminin and the like. Extracellular matrix is known to be degraded by an extracellular matrix protease (matrix metalloproteinase or MMP, referred to below as “MMP”). MMP is an enzyme which is expressed in the course of tissue generation and differentiation via repeated cell division of a fertilized egg, and is also closely associated with invasion and metastasis of cancer. Degradation of extracellular matrix around cancer cells and in the vascular basal membrane is a necessary process for invasion and metastasis of cancer.

### DISCLOSURE OF INVENTION

Thus, invasion and metastasis of cancer can be prevented by inhibiting the expression of MMP, but few drugs have been developed which are highly safe and which inhibit the expression of MMP. There is a need for such drugs.

The present inventors have made the unexpected discovery that menatetrenone (Vitamin K-II) inhibits the expression of MMP. It is an object of the present invention to provide a highly safe drug that has the effect of inhibiting expression of MMP and suppressing cancer cell proliferation.

The invention of this application provides (1) a MMP expression inhibitor comprising, as an active ingredient, menatetrenone or a pharmacologically acceptable salt thereof, or a hydrate thereof; (2) the MMP expression inhibitor according to the item (1) wherein the MMP is selected from the group consisting of MMP-1, MMP-3, MMP-7 or MMP-14.; (3) a uPA expression inhibitor comprising, as an active ingredient, menatetrenone, a pharmacologically acceptable salt thereof, to or a hydrate thereof; (4) an inhibitor of cancer metastasis and invasion comprising, as an active ingredient, menatetrenone or a pharmacologically acceptable salt thereof, or a hydrate thereof; (5) the inhibitor of cancer metastasis and invasion according to item (4) wherein the cancer is hepatic cancer; (6) an AP-1 activity inhibitor comprising, as an active ingredient, menatetrenone or a pharmacologically acceptable salt thereof, or a hydrate thereof; (7) an Ets-1 expression inhibitor comprising, as an active ingredient, menatetrenone or a pharmacologically acceptable salt thereof, or a hydrate thereof; (8) a prognosis improver for cancer therapy comprising, as an active ingredient, menatetrenone or a pharmacologically acceptable salt thereof, or a hydrate thereof; (9) a method for preventing cancer cell metastasis comprising administering an effective dose of menatetrenone or a pharmacologically acceptable salt thereof, or a hydrate thereof in order to inhibit expression of MMP; (10) an expression promoter for CDK inhibitor p16, p21 or p27 comprising, as an active ingredient, menatetrenone or a pharmacologically acceptable salt thereof, or a hydrate thereof.

### BRIEF DESCRIPTION OF THE DRAWINGS

FIG. 1(a) shows a graph showing the relationship between added amount of menatetrenone and proliferation of various hepatic cancer cells. FIG. 1(b) shows RT-PCR results illustrating the relationship between added amount of menatetrenone and CDK inhibitor expression in various hepatic cancer cells. FIG. 1(c) shows Western blot test results illustrating the relationship between added amount of menatetrenone and CDK inhibitor expression in various cancer cells.

FIG. 2 shows cell cycles upon addition of menatetrenone to various hepatic cancer cells.

FIG. 3 shows results for suppression of hepatic cancer cell invasion upon addition of menatetrenone to hepatic cancer cells.

FIG. 4 shows RT-PCR results for expression of various invasion-related factors when menatetrenone was added to various hepatic cancer cells.

FIG. 5 shows Western blot results for expression of various invasion-related factors when menatetrenone was added to various hepatic cancer cells.

FIG. 6 shows gel shift assay results for transcription factor activation when menatetrenone was added.

FIG. 7 shows RT-PCR results for the effect on activity of various MMP promoters when menatetrenone was added to hepatic cancer cells.

FIG. 8 shows RT-PCR results for the effect of menatetrenone on expression of cancer invasion and metastasis related genes induced by TPA.

FIG. 9 shows Western blot results for the effect of menatetrenone on expression of cancer invasion and metastasis related proteins induced by TPA.

### BEST MODE FOR CARRYING OUT THE INVENTION

Next, embodiments of the present invention are explained. The following embodiments are examples for explaining the present invention, and it is not intended that the present invention be limited only to these embodiments. The present invention can be implemented in various modes without departing from the spirit and the scope of the invention.

Menatetrenone has the chemical name 2-methyl-3-tetraprenyl-1,4-naphthoquinone. Its structural formula is as follows.

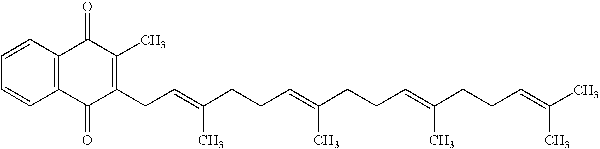

Menatetrenone is a yellow crystalline or oily substance which is odorless and tasteless, and easily decomposed by light. It dissolves very little in water. Menatetrenone is also known as vitamin K-II, and pharmacologically it is involved in carboxylation reaction during conversion of glutamic acid residues to bioactive γ-carboxyglutamic acid in the process of protein synthesis of blood coagulation factors (prothrombin, VII, IX, X), promoting synthesis of normal prothrombin and the like in the liver and producing pharmacologically hemostasis by activating the body's hemostatic mechanism.

Examples of the “pharmacologically acceptable salt” used in the present invention include salts with inorganic acids, salts with organic acids, salts with inorganic bases, salts with organic bases, salts with acidic or basic amino acids and the like. Salts are formed with an appropriate ratio of between 0.1 and 5 molecules of the salt or base, based on 1 molecule of the compound.

Preferable examples of salts with inorganic acids include salts with hydrochloric acid, hydrobromic acid, sulfuric acid, nitric acid, phosphoric acid and other salts. Preferable examples of salts with organic acids include salts with acetic acid, succinic acid, fumaric acid, maleic acid, tartaric acid, citric acid, lactic acid, stearic acid, benzoic acid, methanesulfonic acid, p-toluenesulfonic acid and the like.

Preferable examples of salts with inorganic bases include sodium salts, potassium salts and other alkaline metal salts, calcium salts, magnesium salts and other alkaline earth metal salts and aluminum salts, ammonium salts and the like. Preferable examples of salts with organic bases include salts with diethylamine, diethanolamine, meglumine, N,N′-dibenzylethylenediamine and the like.

Preferable examples of salts with acidic amino acids include salts with aspartic acid, glutamic acid and the like, while preferable examples of salts with basic amino acids include salts with arginine, lysine, ornithine and the like.

The menatetrenone that is an active ingredient of the drug according to the present invention may be an anhydride or may form a hydrate. There may be crystal polymorphism in the menatetrenone but this is not a limitation and the crystal form may be uniform or a mixture of multiple forms. Moreover, a metabolite produced by degradation of the menatetrenone of the present invention in vivo is also encompassed by the claims of the present invention.

The menatetrenone used in the present invention may be manufactured by the known methods, and as a typical example it may be easily manufactured by the methods disclosed in Japanese Patent Application Laid-Open No. S49-55650, or may be easily obtained from a chemical manufacturer. Menatetrenone can also be obtained as a capsule, injection or other preparation. For the drug according to the present invention, menatetrenone may be used as is or may be mixed with known pharmacologically acceptable carriers and the like (for example excipients, binders, disintegrators, lubricants, colorants, flavorings, stabilizers, emulsifiers, absorption promoters, surfactants, pH adjusters, preservatives, antioxidants and the like) and components commonly used as raw materials for drug preparations, and formulated by ordinary methods. Components such as vitamins, amino acids and the like can also be included as necessary. Examples of forms of preparation include tablets, powders, grains, granules, capsules, syrups, suppositories, injections, ointments, poultices and the like.

In the present invention there are no particular limitations on the form of administration of menatetrenone, but it is preferably administered orally. Menatetrenone capsules can be obtained under the trade names Kaytwo capsules (manufactured by Eisai Co. Ltd.) and Glakay capsules (manufactured by Eisai Co. Ltd), syrup under the trade name Kaytwo syrup (manufactured by Eisai Co. Ltd), and injections under the trade name Kaytwo N injection (manufactured by Eisai Co. Ltd).

A preferable dosage of menatetrenone is generally 1 to 500 mg/day, preferably 10 to 200 mg/day, more preferably 30 to 135 mg/day.

As mentioned above, “MMP” is an abbreviation for matrix metalloproteinase, and includes for example MMP-1, MMP-2, MMP-3, MMP4, MMP-7, MMP-9, MMP-10, MMP-11, MMP-12, MMP13, MMP-14 and the like, but is not particularly limited thereby in these specifications. The term “MMP” refers to MMP in general.

Moreover, uPA (urokinase plasminogen activator) is a kind of plasminogen activator (PA), and is an enzyme involved in fibrinolytic reaction and also in cancer invasion and metastasis.

### EXAMPLES

Examples and reference examples are given below to indicate the advantageous effects of the present invention, but these are only examples and the present invention is not limited to the following specific examples under any circumstances. The skilled person in the art can implement the present invention while appropriately changing the conditions described in the examples given below, and such changes are included in the scope of the claims of this application.

The present inventors performed the following tests to study the effects of menatetrenone on (1) proliferation and (2) invasion and metastasis of cancer cells.

(Investigation of Inhibitory Effect of Menatetrenone on Cancer Cell Proliferation)

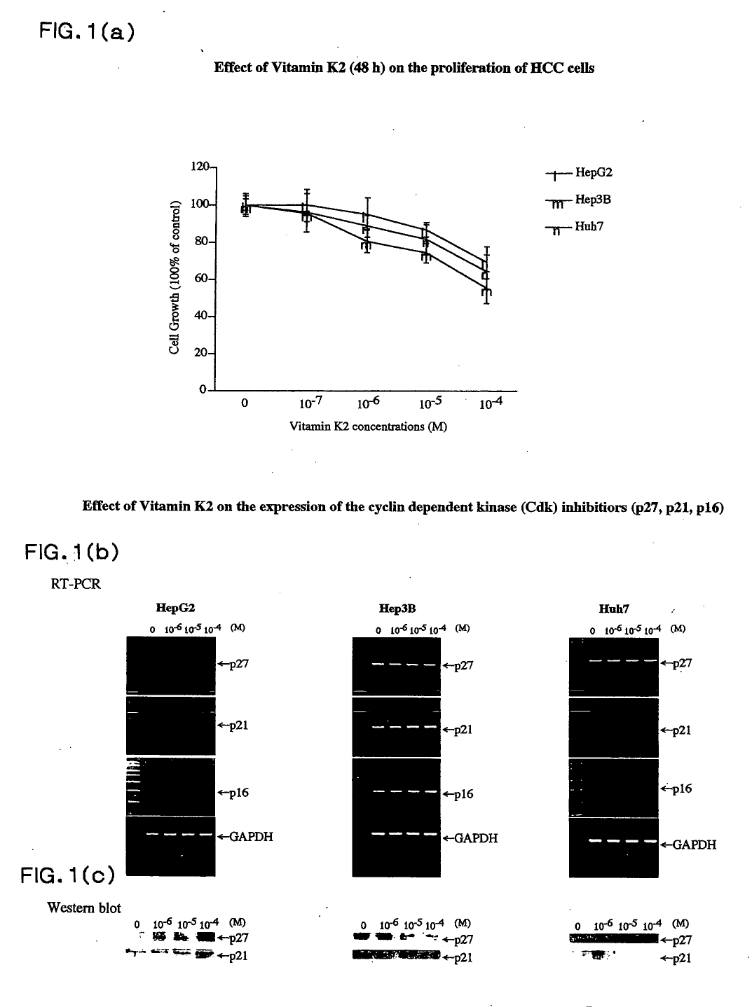

Menatetrenone was added at concentrations of 0 M, 10⁻⁶ M, 10⁻⁵ M and 10⁻⁴ M to HepG2, Huh7, Hep3B and HLE hepatic cancer cell lines, and cell proliferation was studied by WST assay after 48 hours. The results are shown in FIG. 1(a). As shown in FIG. 1(a), the cells to which menatetrenone was added had their proliferation dose-dependently inhibited in comparison with the control hepatic cancer cells which had no menatetrenone added to any cells.

(Investigation of Expression of Cell Cycle Regulator Genes)

To investigate the mechanism of inhibition of cell proliferation, genes involved in promoting the cell cycle were focused on, and changes caused by addition of menatetrenone in the amount of expression of CDK inhibitors p21, p27 and p16 which inhibit cyclin dependent kinase (CDK), an enzyme which promotes the cell cycle, were analyzed by the RT-PCR and Western blot methods.

(RT-PCR Method)

In this test, menatetrenone was added at concentrations of 0 M, 10⁻⁶ M, 10⁻⁵ M and 10⁻⁴ M to HepG2, Huh7, Hep3B and HLE hepatic cancer cell lines, RNA was collected after 48 hours, and 1 μg of total RNA was subjected to RT-PCR to investigate expression of p21, p27 and p16. RT-PCR was performed on GAPDH (glyceraldehyde 3-phosphate dehydrogenase) as a control. The results are shown in FIG. 1(b).

(Western Blot Method)

In this test, menatetrenone was added at concentrations of 0 M, 10⁻⁶ M, 10⁻⁵ M and 10⁻⁴ M to HepG2, Huh7, Hep3B and HLE hepatic cancer cell lines, proteins were extracted from the various cells, subjected to SDS-PAGE electrophoresis, blotted on PVP membranes and then incubated with p21 and p27 antibodies, and p21 and p27 protein expression was investigated by ECL. The results are shown in FIG. 1(c).

FIGS. 1(b) and 1(c) show increases in p27 and p21 mRNA in HepG2 cells which are dependent on the added dose of menatetrenone. In Hep3B cells, a slight increase in p21 and p16 was seen but no change in p27. In Huh7 cells there were no changes in these expressions.

(Investigation of Cell Cycle)

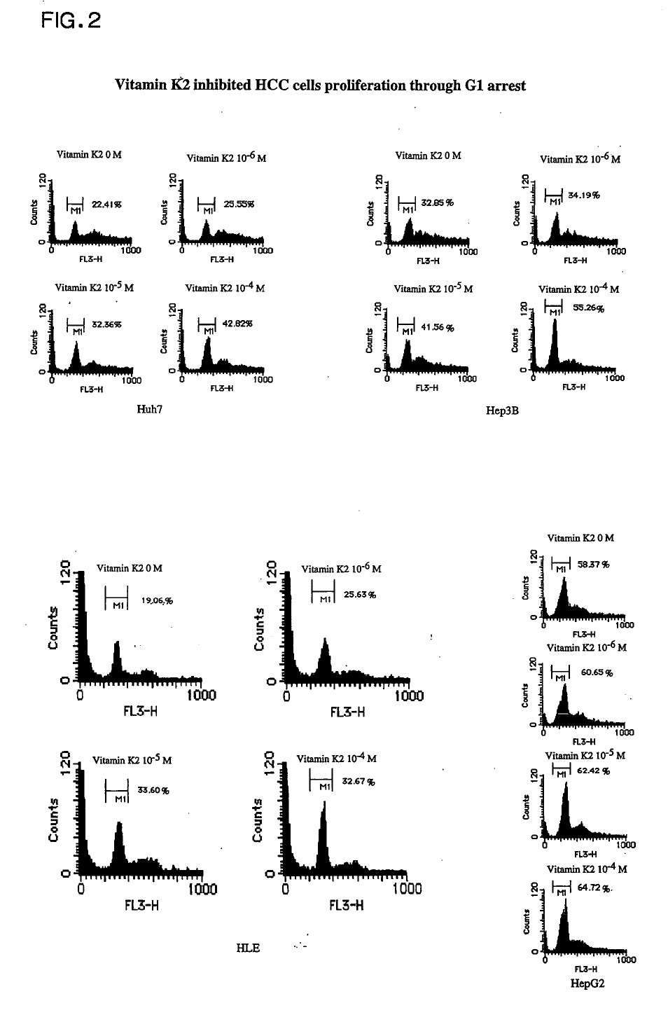

To investigate the effects of menatetrenone on the cell cycles of cancer cells, distributions according to DNA amount in hepatic cancer cells to which menatetrenone had been added were analyzed by flow cytometry (FACS). In this test, menatetrenone was added at concentrations of 0 M, 10⁻⁶ M, 10⁻⁵ M and 10⁻⁴ m to HepG2, Huh7, Hep3B and HLE hepatic cancer cells, and changes in the cell cycles were observed by FACS 48 hours later. The results are shown in FIG. 2. As shown in FIG. 2, the proportion of G1-phase cells increased dose-dependently in all cells due to addition of menatetrenone, while G2-phase cells decreased. This suggests that menatetrenone inhibits progression from the G1 phase to the S phase in the cell cycle of hepatic cancer cells, so it appears that menatetrenone inhibits hepatic cancer cell proliferation by inducing G1 arrest.

(Investigation of Cancer Cell Invasion and Metastasis)

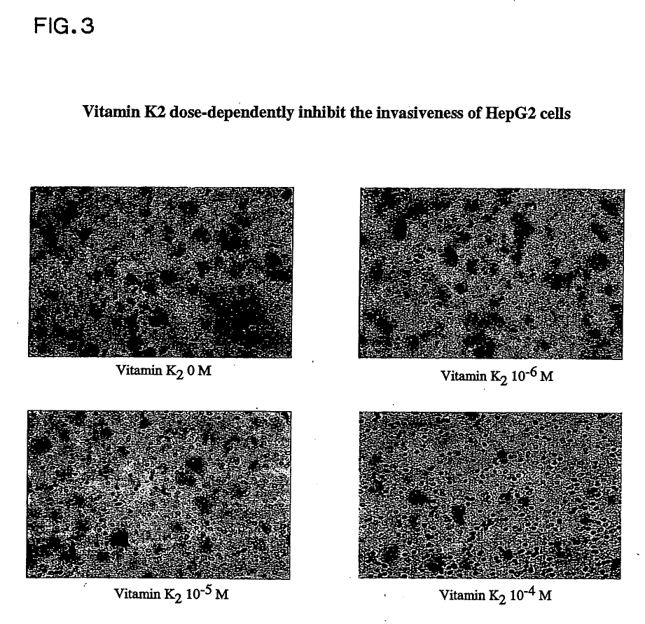

To investigate the effect of menatetrenone in inhibiting invasion and metastasis of cancer cells, menatetrenone was added to hepatic cancer cells (HepG2) at concentrations of 0 M, 10⁻⁶ M, 10⁻⁵ M and 10⁻⁴ M, and ability of the cancer cells to invade Matrigel was measured. In this test, cancer cells were spread on Matrigel which had been coated on the upper chamber of a double chamber, menatetrenone was added thereto. NIH3T3 cells were spread on the lower chamber as a feeder. The number of cells that had passed through the Matrigel and migrated to the bottom of the upper chamber after 24 hours was observed microscopically. The results are shown in FIG. 3. As shown in FIG. 3, the greater the concentration of added menatetrenone the smaller the number of cells which migrated to the bottom of the upper chamber. These results indicate that menatetrenone dose-dependently inhibits the ability of cancer cells to invade Matrigel.

The effects of menatetrenone on MMP expression were investigated by (1) RT-PCR test, (2) Western blotting test, (3) gel shift assay, (4) MMP gene promoter activity test and (5) testing of gene expression with TPA induction.

(RT-PCR Method)

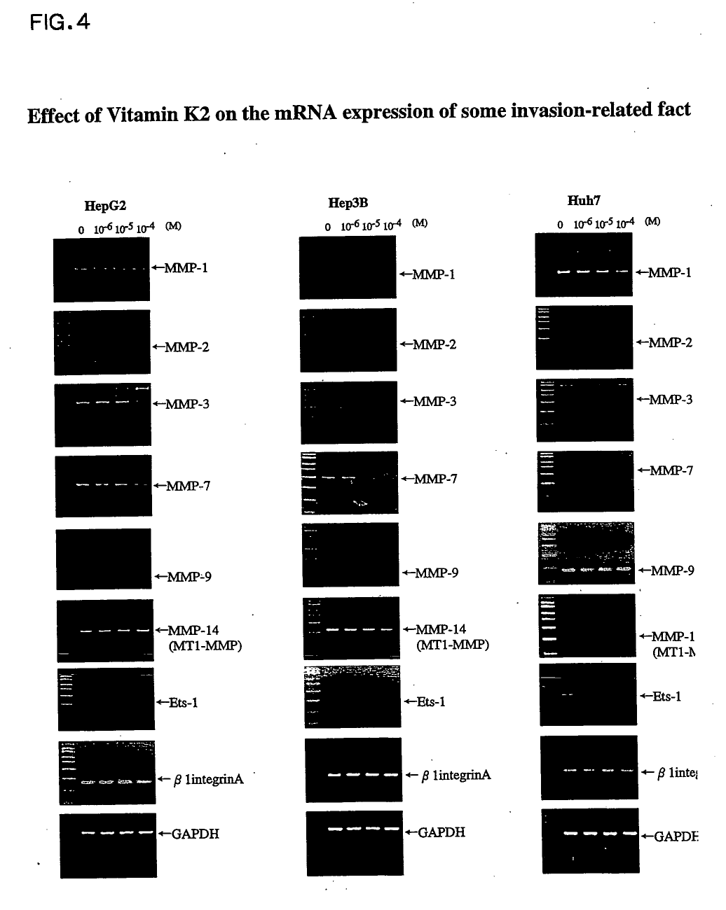

Test was performed by RT-PCR to investigate MMP expression. This test was to investigate expression of various MMPs (MMP-1, MMP-2, MMP-3, MMP-7, MMP-9 and MMP-14 (MT1-MMP)) and of transcription factor Ets-1 that affect MMP expression and the extracellular matrix receptor β1 integrin when menatetrenone was added to various hepatic cancer cells (HepG2, Hep3B, Huh7). The results are shown in FIG. 4. As shown in FIG. 4, there were some differences in the kinds of MMPs which were expressed by the hepatic cancer cells, but expression of mRNA of MMP-1, MMP-3, MMP-7 and MMP-14 was dose-dependently inhibited by addition of menatetrenone. Moreover, expression of the transcription factor Ets-1 was also dose-dependently inhibited, but there were no changes in expression of β1 integrin and GAPDH mRNA.

(Western Blot Method)

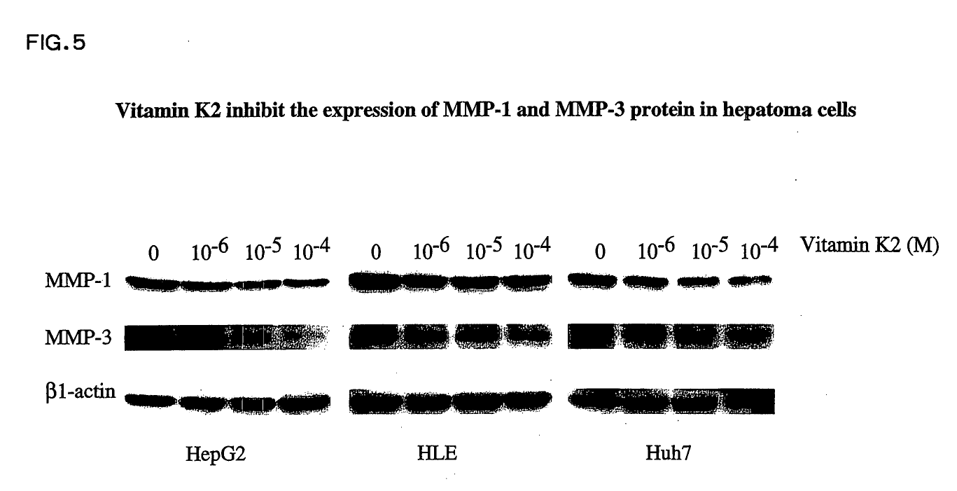

Testing was performed by Western blot to investigate MMP expression. In this test protein expressions of MMP-1 and MMP-3 were investigated when menatetrenone was added to various hepatic cancer cells (HepG2, Hep3B, Huh7). The results are shown in FIG. 5. As shown in FIG. 5, protein expressions of MMP-1 and MMP-3 were inhibited as a result of menatetrenone addition.

(Gel Shift Assay)

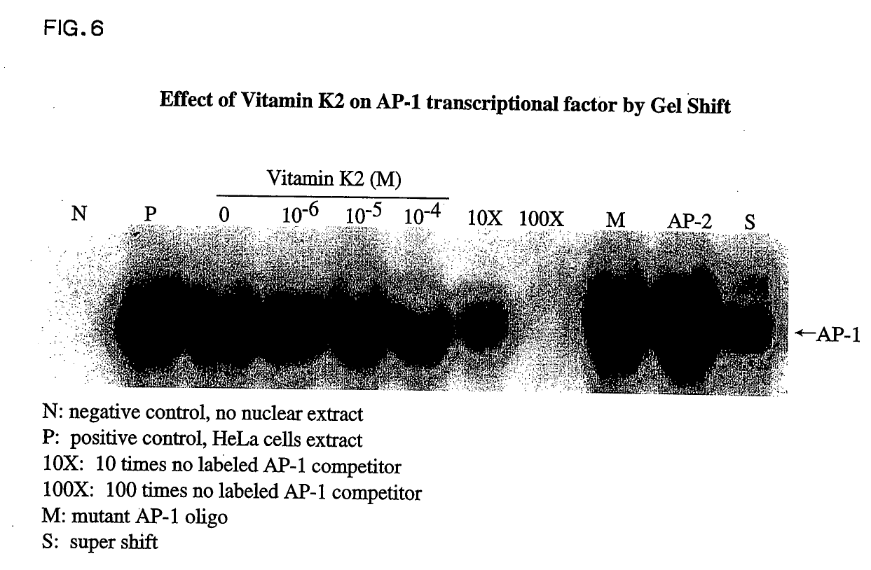

MMP have binding sites for transcription factors AP-1, Ets-1, Tcf/Lef and the like at common to their promoter regions and which are known to regulate MMP expression. Gel shift assay was performed to investigate whether the binding activity of transcription factors AP-1 and Tcf/Lef, which regulate MMP expression, is affected by addition of menatetrenone. In this test menatetrenone was added to HepG2 hepatic cancer cells, nuclear proteins were extracted after 24 hours and reacted with double-stranded DNA having binding sites for AP-1 and Tcf/Lef labeled with ³²P, and gel shift assay was performed. The results are shown in FIG. 6. As shown in FIG. 6, menatetrenone did not affect the binding activity of Tcf/Lef, but dose-dependently inhibited the binding activity of AP-1. In the case of binding of excess unlabeled AP-1 probe, the band was dose-dependently reduced (“10×”, “100×” in the Figure), while in the case of a mutant AP-1 probe (“M” in the Figure) there were no changes. Reduction and shifting of the band was also seen with anti-c-fos antibodies (“S” in the Figure). These results show that menatetrenone specifically inhibits binding of AP-1.

(MMP Gene Promoter Activity Test)

Next, promoter reporter genes for MMP-1, MMP-3 and MMP-7 were introduced into Huh7 hepatic cancer cells, and the effect of menatetrenone on the activity of these MMP promoters was investigated. The MMP-1, MMP-3 and MMP-7 promoter-luciferase reporter plasmids used were prepared by replacing the reporter gene part of the MMP promoter-CAT reporter plasmid reported in Ozaki I, Mizuta T, Zhang H, Yoshimura T, Kawazoe S, Eguchi Y, Yasutake T, Hisatomi A, Sakai T and Yamamoto K, “Induction of multiple matrix metalloproteinase genes in human hepatocellular carcinoma by hepatocyte growth factor via a transcription factor Ets-1,” Hepatol. Res. 27, 288-300. The various MMP-luciferase plasmids were introduced into Huh7 hepatic cancer cells using lipofectamine, menatetrenone was added at various concentrations, and after 48 hours the cells were collected and luciferase activity measured to investigate the effects of menatetrenone on MMP-1, MMP-3 and MMP-7 gene promoter activity.

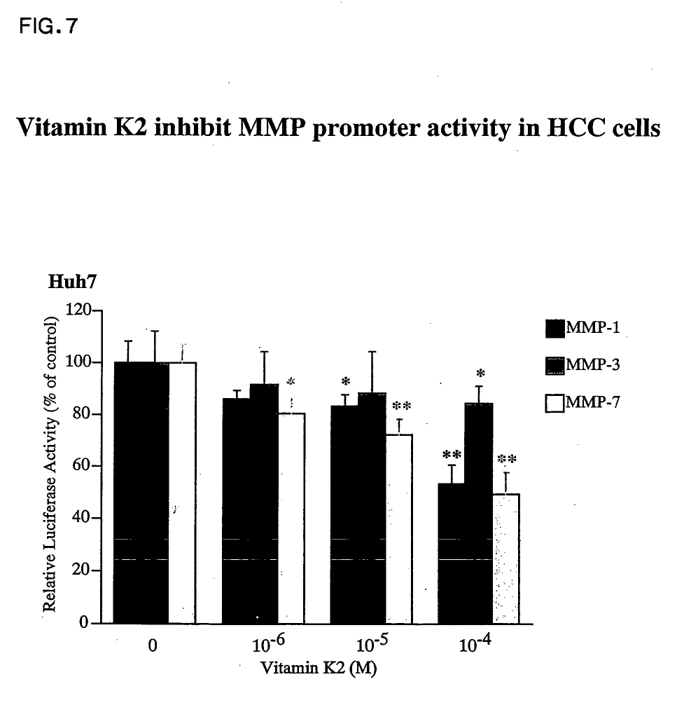

The results are shown in FIG. 7. As shown in FIG. 7, MMP-1, MMP-3 and MMP-7 promoter activity in Huh7 cells was dose-dependently inhibited by addition of menatetrenone. In particular, MMP-1 and MMP-7 activity decreased almost 50%. This indicates that suppression of MMP expression by menatetrenone occurs due to suppression of promoter activity for these genes.

(Test of Gene Expression with TPA Induction)

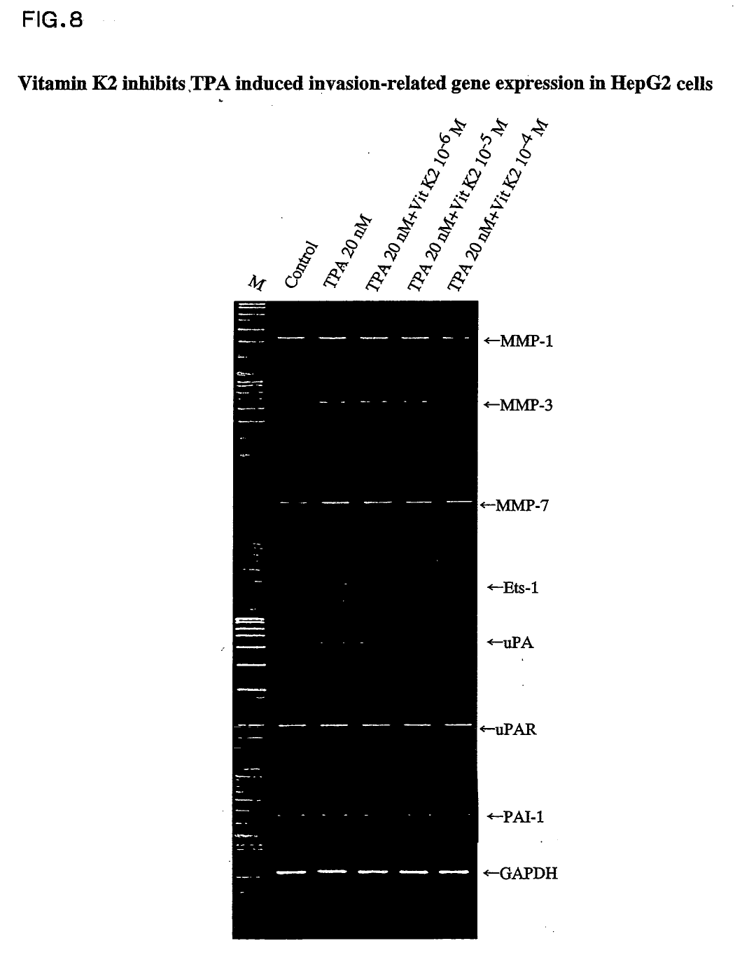

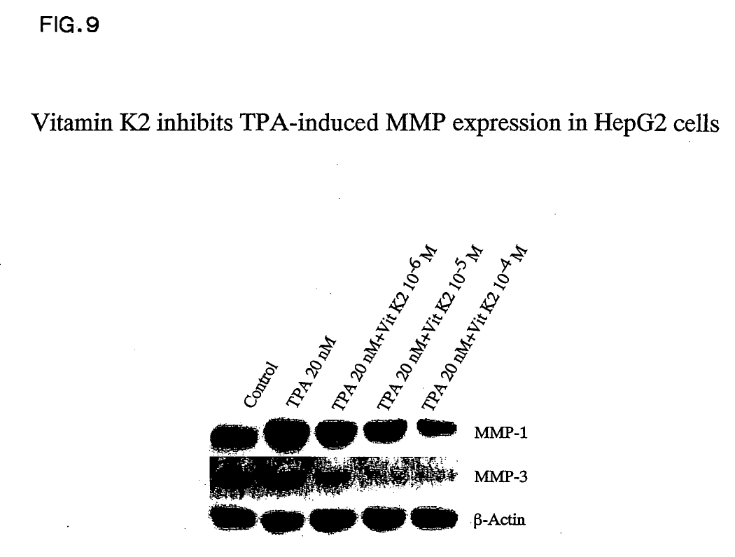

MMP and Ets-1, the expression of which is inhibited by menatetrenone, are known to be genes that are induced by 12-O-tetradecanoylphorbol-13-acetate (TPA) as well as uPA (urokinase plasminogen activator) and the like. In order to discover whether menatetrenone controls expression of genes activated by TPA, TPA was added to HepG2 cultured hepatic cancer cells at a concentration of 20 nM, menatetrenone was then added thereto, and RT-PCR and Western blot were performed to investigate whether expression of the aforementioned gene expression of which was induced by TPA was affected. The results are shown in FIGS. 8 and 9. As shown in FIG. 8, mRNA expression of MMP-1, MMP-3, MMP-7, Ets-1 and uPA increased when 20 nM of TPA was added to HepG2 hepatic cancer cells. On the other hand, the uPA receptor uPAR and the uPA inhibitor PAI were not affected. When menatetrenone was added thereto, TPA-induced mRNA expression of MMP-1, MMP-3, MMP-7, Ets-1 and uPA was dose-dependently inhibited. Moreover, as shown in FIG. 9, menatetrenone also dose-dependently inhibited expression of MMP-1 and MMP-3 at the protein level. These results indicate that in hepatic cancer cells menatetrenone inhibits the TPA-induced expression of MMP, Ets-1, uPA and other genes involved in invasion and metastasis.

### INDUSTRIAL APPLICABILITY

According to the present invention, it is possible to suppress invasion-and metastasis of cancer cells because menatetrenone inhibits expression of the transcription factor Ets-1 and binding activity of AP-1, and inhibits and prevents expression of both MMP which degrade the extracellular matrix and uPA, an enzyme involved in invasion and metastasis of cancer as well as MMP.

We claim:

1. A MMP expression inhibitor comprising, as an active ingredient, menatetrenone or a pharmacologically acceptable salt thereof, or a hydrate thereof.

2. The MMP expression inhibitor according to claim 1 wherein the MMP is selected from the group consisting of MMP-1, MMP-3, MMP-7 or MMP-14.

3. A uPA expression inhibitor comprising, as an active ingredient, menatetrenone, a pharmacologically acceptable salt thereof, or a hydrate thereof.

4. An inhibitor of cancer metastasis and invasion comprising, as an active ingredient, menatetrenone or a pharmacologically acceptable salt thereof, or a hydrate thereof.

5. The inhibitor of cancer metastasis and invasion according to claim 4 wherein the cancer is hepatic cancer.

6. An AP-1 activity inhibitor comprising, as an active ingredient, menatetrenone or a pharmacologically acceptable salt thereof, or a hydrate thereof.

7. An Ets-1 expression inhibitor comprising, as an active ingredient, menatetrenone or a pharmacologically acceptable salt thereof, or a hydrate thereof.

8. A prognosis improver for cancer therapy comprising, as an active ingredient, menatetrenone or a pharmacologically acceptable salt thereof, or a hydrate thereof.

9. A method for preventing cancer cell metastasis comprising administering an effective dose of menatetrenone or a pharmacologically acceptable salt thereof, or a hydrate thereof in order to inhibit expression of MMP.

10. An expression promoter for CDK inhibitor p16, p21 or p27 comprising, as an active ingredient, menatetrenone or a pharmacologically acceptable salt thereof, or a hydrate thereof.
